(19) United States
(12) Patent Application Publication
(10) Pub. No.: US 2012/0172417 A1
(43) Pub. Date: Jul. 5, 2012

(54) System And Method For Identifying And Applying Peripheral Treatment Mechanisms For Disease

(75) Inventor: Stefan Keslacy, Syracuse, NY (US)

(73) Assignee: SYRACUSE UNIVERSITY, Syracuse, NY (US)

(21) Appl. No.: 13/238,647

(22) Filed: Sep. 21, 2011

Related U.S. Application Data
(60) Provisional application No. 61/429,326, filed on Jan. 3, 2011.

(51) Int. Cl. A61K 31/7088; C40B 30/00; A61P 25/00; A61P 25/28; A61P 3/04; A61P 3/10

(52) U.S. Cl. 514/44 R; 506/7

(57) ABSTRACT

A method and system for the identification and application of biological targets for peripheral treatment of diseases. Existing cellular mechanisms or pathways are exploited to identify novel genes or other molecule candidates that will be used to treat disease via a peripheral treatment system. Using the method, a novel Alzheimer's disease target is identified and used to treat an animal Alzheimer's disease model via peripheral expression of that target.

### CROSS-REFERENCE TO RELATED APPLICATIONS

The present application claims priority to U.S. Provisional Patent Application Ser. No. 61/429,326 filed on Jan. 3, 2011 and entitled “System and Method for Prevention of Neurodegenerative Diseases,” the entirety of which is hereby incorporated herein by reference.

### BACKGROUND OF THE INVENTION

1. Field of the Invention

The present invention relates to peripheral-directed methods for the treatment of disease, and, more particularly, to methods and systems for the identification and application of biological targets for peripheral treatment of disease.

2. Description of the Related Art

Traditionally, medical research focuses on only one component of a disease rather than on a systemic view of disease. For example, many new drugs are targeted to, and are meant to act on, a single organ or body system. Direct targeting of disease can be challenging, however, when treatments are unable to reach the target. For example, in the case of disease related to the brain, treatments must be able to effectively pass the blood-brain barrier in order to reach the site of the disease. Direct targeting also ignores the system-wide effects of disease. Many diseases have primary or secondary effects outside the main target organ or system and thus can also benefit from treatment.

Alzheimer's disease (“AD”) is one example of a disease that could benefit from a peripheral treatment approach. Brain health is modulated by various peripheral and central factors. For instance, there is increasing evidence that risk factors such as hypertension, diabetes, and obesity are important in AD etiology. A common feature that contributes to these conditions, conceptualized as the metabolic syndrome, is systemic inflammation, which contributes to brain inflammation and correlates with neurodegeneration. If a deleterious environment could affect the brain, a beneficial milieu could improve AD. Recent experiments that aimed to reduce Aβ deposition by increasing its peripheral clearance were promising. Peripheral administration of a monoclonal antibody directed against Aβ markedly reduced Aβ deposition in the brain. Thus, peripheral immune-based therapeutic strategies may be useful for AD, but more research is needed in order to identify safer alternatives.

### BRIEF SUMMARY OF THE INVENTION

It is therefore a principal object and advantage of the present invention to identify biological targets for the treatment mechanisms of disease.

It is another object and advantage of the present invention to provide a method for the identification of biological targets for the treatment mechanisms of disease.

It is yet another object and advantage of the present invention to identify and provide targets for the treatment mechanisms of disease using peripheral organs or systems.

Other objects and advantages of the present invention will in part be obvious, and in part appear hereinafter.

In accordance with the foregoing objects and advantages, as aspect of the invention provides a method of identifying an agent for the treatment of a disease in a subject. The method comprises the steps of: (i) exposing the subject to an exercise regimen, wherein the exercise regimen results in an improvement of a hallmark of the disease; (ii) profiling the expression of a gene in the subject; and (iii) determining whether the expression of the gene is altered after exposure to the exercise regimen. Other aspects of the method further comprise one or more of the steps of: (iv) generating a vector comprising at least a portion of the identified gene; (v) transfecting the vector into a cell of a second subject; and (vi) determining whether transfection of the vector into a cell of a second subject results in an improvement of one or more hallmarks of the disease. According to one embodiment, the expression of the gene may be up-regulated after exposure to the exercise regimen, or may be down-regulated after exposure to the exercise regimen. Preferably, the cell is remote from the brain of the subject, including a cell such as a muscle cell. Also, the subject is preferably a mammal such as a human.

According to a second aspect of the invention is provided a method for the treatment of a condition. The method comprises the steps of: (i) identifying a target for the treatment of the condition, wherein the target is identified by a method comprising the steps of: (a) exposing a first subject to an exercise regimen, wherein the exercise regimen results in an improvement of a hallmark of the condition; (b) profiling the expression of a gene in the first subject; and (c) identifying the gene as the target if the expression is altered after exposure to the exercise regimen; (ii) generating a vector comprising the identified gene; and (iii) transfecting the vector into a cell of a subject suffering from the condition. According to one embodiment, the expression of the gene may be up-regulated after exposure to the exercise regimen, or may be down-regulated after exposure to the exercise regimen. Preferably, the cell is remote from the brain of the subject, including a cell such as a muscle cell. Also, the subject is preferably a mammal such as a human. The condition may be selected from a group consisting of dementia, obesity and diabetes, or may be any other condition.

According to a third aspect of the invention is provided a method of treating cognitive decline in a subject using PPARγ coactivator 1-alpha (“PGC-1α”) protein. The method comprises the step of expressing a gene sequence in a cell of the subject whereby the PGC-1α protein is produced. Preferably, the cell is remote from the brain of the subject, including a cell such as a muscle cell.

According to a fourth aspect of the invention is provided a method of treating cognitive decline in a subject using PPARγ coactivator 1-alpha (“PGC-1α”) protein. The method comprises the step of increasing an activity of PGC-1α in a cell of the subject. Preferably, the cell is remote from the brain of the subject, including a cell such as a muscle cell. According to one embodiment, increasing an activity of PGC-1α comprises increasing the expression of PGC-1α in a cell of the subject.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a high-level schematic of a method to identify one or more gene candidates according to one embodiment;

FIG. 2 is a high-level schematic of a method to identify gene candidates in a mouse model according to one embodiment;

FIG. 3 is a high-level schematic of a method to use identified gene candidates to treat a disease condition according to one embodiment;

FIG. 4 is a high-level schematic of a method to use a gene candidate identified in a mouse model to treat the symptoms of a mouse model of Alzheimer's disease according to one embodiment;

FIG. 5 is a series of micrographs and charts representing Aβ plaque deposition (fluorescence) in the brains of AD mice without (A) and (B) with exercise exposure, with (C) average plaque count and (D) average densitometry comparing sedentary (“SED”) and exercise (“EX”) AD animals;

FIG. 6 are charts illustrating the effect of exercise on AD mice behavior, where odorant-induced reflexive behavioral response comparing (A) wild type (WT) and AD (APP/PS1) sedentary and (B) trained animals;

FIG. 7A is a schematic illustrating gene regulation following exercise in skeletal muscle of AD animals, specifically a heat map depicting 69 genes regulated by AD that are not different from wild type animals following exercise;

FIG. 7B is a schematic showing the most important pathway involved in the effect of exercise on AD in skeletal muscle based on the information depicted in FIG. 7A;

FIG. 8A is a photograph illustrating in vivo gene delivery in skeletal muscles using electroporation, showing bioluminescence in an animal five days after PcDNA-firefly luciferase injection in both legs;

FIG. 8B is a photograph illustrating in vivo gene delivery in skeletal muscles using electroporation, showing bioluminescence in the same animal as FIG. 8A, but 30 days after PcDNA-firefly luciferase injection in both legs; and

FIG. 9 is a series of micrographs and charts representing Aβ plaque deposition following gene delivery in the muscle APP/PS1 animals, where (A) shows PcDNA injection and (B) shows PcDNA/PGC-1, quantification of (C) plaque numbers, and (D) densitometry comparing PcDNA control and PcDNA/PGC-1.

### DETAILED DESCRIPTION OF THE INVENTION

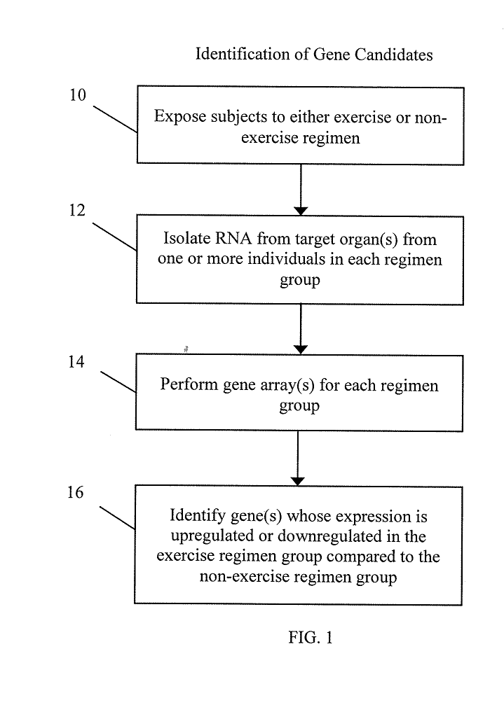

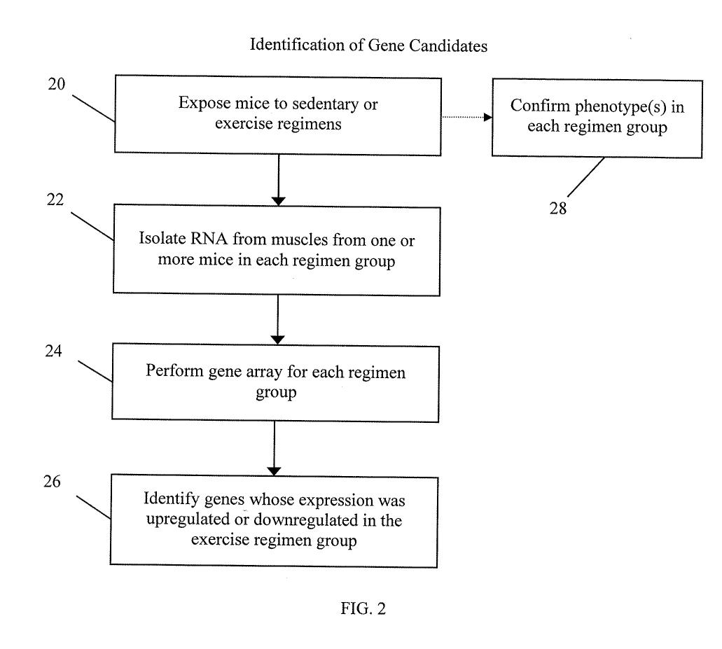

Referring now to the drawings, wherein like reference numerals refer to like parts throughout, there is seen in FIG. 1 a flowchart of a method for the identification of one or more gene candidates for the treatment of a condition or disease. At step 10, subjects are exposed to either an exercise or non-exercise regimen. The exercise regimen can be anywhere along a scale from extremely intense to extremely moderate or weak, depending upon the requirements of the experiments and the abilities of the subject. The regimens can be implemented for a variable amount of time ranging from minutes to months or years.

At step 12, RNA is obtained from one or more target organs from one or more individuals in each regimen group. For example, RNA can be isolated from cells obtained from an individual in the exercise regimen group before and/or after an exercise or non-exercise regiment, as well as from an individual in the non-exercise regimen group. At step 14, gene profiling is performed using the obtained RNA according to standard protocols known in the art. At step 16, the genes whose expression is either upregulated or downregulated in the exercise regimen group compared to the non-exercise regimen group are identified. One or more of these genes are then examined according to other methods described herein to determine the viability of that gene to treat the condition or disease of interest.

FIG. 2 is a flowchart of a method for the identification of a gene candidate for the treatment of cognitive symptoms in an Alzheimer's disease mouse model. At step 20, the mice—optionally comprising APP/PS1 mice or another suitable AD model—are divided into two or more groups such as wild-type (control, non-transgenic animals), sedentary (typical or normal physical activity (i.e., no physical activity intervention)), and exercise (increased physical activity compared to normal).

At optional step 28, the desired phenotype is confirmed by comparing the phenotypes of mice in the different groups. For the AD mouse model, for example, the mice in the exercise group are examined to determine an improvement in AD hallmarks (such as plaque characteristics and behavioral aspects) compared to the wild-type or sedentary animals. For other models, such as obesity or diabetes, an improvement in obesity or diabetes hallmarks (weight, improved glucose tolerance test, decreased insulin resistance, health, etc.) in the exercise animals compared to the wild-type or sedentary animals is determined.

At step 22, following exposure to the regimen for a certain period of time ranging from hours to years, RNA is isolated from mice in each group using well-known methods and/or protocols. According to one embodiment, RNA is isolated from the soleus muscles of the mice. At step 24, a gene array is performed using the isolated RNA and well-known methods and/or protocols.

Finally, at step 26, the gene profile is examined to identify all those genes that are upregulated or downregulated in the exercise regimen group compared to the non-exercise regimen group. One or more of these genes are then examined according to other methods described herein to determine the viability of that gene to treat the condition or disease of interest.

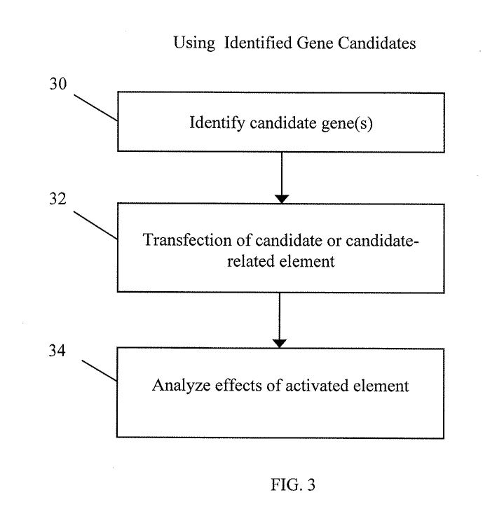

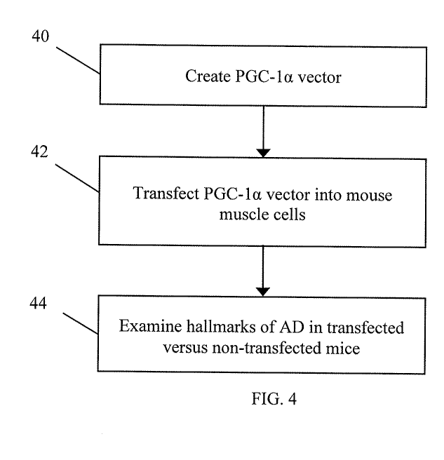

There is seen in FIG. 3 a flowchart of a method of using an identified gene to prevent and/or treat a condition or disease according to one embodiment. At step 30, the candidate gene is identified using any of the methods identified herein, including those depicted in FIGS. 1 and 2. In one embodiment, described in detail below, the gene is identified as one that is significantly up-regulated during conditions that reduce the effect(s) of the disease (i.e. the exercise). In an AD mouse model, for example, intense exercise has been suggested to reduce amyloid plaques deposition. Accordingly, performing gene profiling of mouse muscle tissue from mice that experience intense exercise versus mice that do not exercise will identify genes whose expression is up-regulated, down-regulated, or unaffected by intense exercise. Genes that show a significant change in expression levels in the exercising mice are thus identified as candidates for downstream steps of the process.

Alternatively, the candidate can be identified using a variety of other means, including but not limited to the following: (i) gene profiling of obese versus non-obese animal models to identify candidates for obesity treatments; (ii) gene profiling of arthritic versus non-arthritic animal models to identify candidates for arthritis treatments; (iii) gene profiling of plaque-covered versus non-plaque-covered arterial cells for hypertension and/or artherosclerosis treatments; (iv) gene profiling of cancer versus non-cancer animal models; and (v) gene profiling of insulin-producing cells in diabetic versus non-diabetic or pre-diabetic patients. Many other methods of identifying a treatment candidate are possible.

At step 32, transfection occurs, where transfection is the introduction of a nucleic acid or other agent into one or more cells. This may be transfection of the candidate gene, or may be transfection of an agent that affects the candidate (such as, for example, an activator or inhibitor of either expression or translation). In a preferred embodiment, the nucleic acid or other agent is transfected into a peripheral cell. In one embodiment, described in detail below, the identified gene candidate is introduced into peripheral cells, such as muscle tissue, by electroporation, although other methods of transfection are known.

At optional step 34, the effect of the activated element on the disease is analyzed. This step confirms that the candidate was properly identified, and that inducing or inhibiting the expression of the candidate is able to effectuate a disease treatment. In the example described below, studies are conducted to determine whether expression of the gene candidate mimics the effect of intense exercise (including Aβ plaque reduction and the amelioration of memory and olfactory deficits). If expression of the candidate does mimic the effects of intense exercise, then the candidate was properly identified and is thus a viable candidate for treatment.

### EXAMPLE 1

### Identifying Gene Candidates for Alzheimer's Disease

Effective strategies for preventing/treating Alzheimer's disease (“AD”) have become a public health priority given the baby boomer cohort. An Alzheimer's disease “tsunami” is inevitable in the near future as 16 million Americans will develop the disease by 2050. As people live longer, the incidence of debilitating neurological disorders such as AD is expanding, creating formidable therapeutic challenges. A defining feature of AD is accumulation of amyloid peptides (Aβ) that induces plaque deposition and hyperphosphorylation of a protein (Tau) that induces neurofibrillar tangles within the diseased brain. Converging evidence suggests that Aβ toxicity is the nidus of non-immune inflammatory events in the brain.

Although there is currently no cure for AD, physical activity (referred to herein as exercise) is recognized as one of the most promising new treatments for the disease. Although scientific studies have shown that exercise has beneficial effects on both cognition and quality of life in people suffering from AD, there is a lack of knowledge concerning the cellular mechanism(s) involved in this process.

Research using human subjects has shown that exercise provides benefits for overall health and improvement of cognitive function. Meta-analysis of longitudinal studies reveals that the incidence of dementia and AD decreases with an active lifestyle. During a follow up of more than 6 years, one researcher reported that an exercise frequency of 2-3 times a week is associated with risk reduction of dementia and AD. Increased cardio-respiratory fitness-induced by exercise is thought to, among other things, preserve hippocampus volume in AD patients. Although exercise protocols for AD patients have not been well established and making exercise accessible to elderly adults with dementia is still problematic, understanding the mechanism underlying the beneficial effects of exercise on AD is important and may provide other means of offering new therapeutic targets. Indeed, an alternative to exercise is likely necessary as the majority of AD sufferers are age 65 and older, an age when other chronic diseases are likely to appear that limit exercise such as osteoarthritis and when instauration of a new lifestyle is more challenging.

Skeletal muscle fibers are not only responsible for mechanical functions, but participate in the inflammatory process. After exercise exposure, well-known anti-inflammatory cytokines such as IL-10 and IL-1ra expression have been shown to be produced by skeletal muscle cells (thereby called “myokines”). The release of anti-inflammatory myokines in the systemic circulation may be mediated by transcription factors activation such as Nuclear Factor kappa-B (NF-κB) in skeletal muscle. Further, exercise may create an anti-inflammatory milieu in particular through skeletal muscle-associated interleukin 6 (IL-6) expression which is beneficial for AD.

Ultimately, it was hypothesized that exercise induces changes in genes involved in the inflammatory process at the muscle level that protect the brain in AD, although the genes and pathways involved in this process were completely unknown. Further, it was hypothesized that gene delivery at the muscle level would partially reproduce the effect of exercise, including perhaps the anti-inflammatory effect of exercise, thereby reducing AD-associated biological and cognitive deficiencies.

Results

Effect of Exercise on Amyloid Plaque Deposition

To produce the preliminary results, transgenic mice overexpressing mutant human APP, with the Swedish (K670N, M671L), Florida (1716V), London (V717I) and human PS1 mutations, were utilized. APP/PS1 mice were used because they rapidly develop AD symptoms with accelerated Aβ accumulation. Further, a diminished activity in neurons in the vicinity of Aβ-associated plaques has been recently reported in APP/PS1 mice, suggesting that plaques burden is an essential element of AD-related neurodegeneration. The strain B6SJLF1 was used to produce APP/PS1 as a proper control.

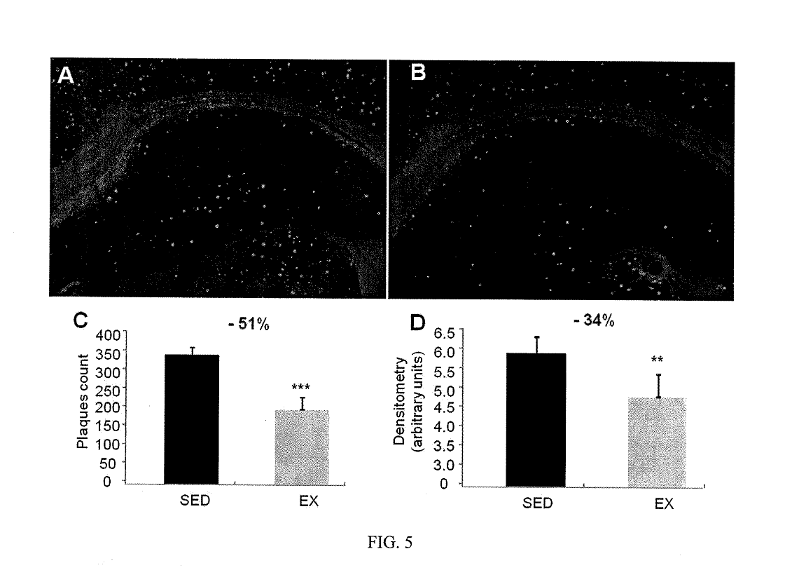

Hallmarks of AD include plaque deposition induced by Aβ, and studies report reduced amyloid load with exercise. The preliminary data show that at 6 months of age, APP/PS1 animals show Aβ plaque deposition within the brain (FIG. 5A) and a month of exercise (via a running wheel) decreases both number (51%) and density (34%) of Aβ plaques (FIG. 5B) (see Table 1 for information about exercise frequency). Together, these results show that exercise is associated with a reduction of Aβ plaque and can be use as positive evidence of the effect of exercise on APP/PS1 mice.

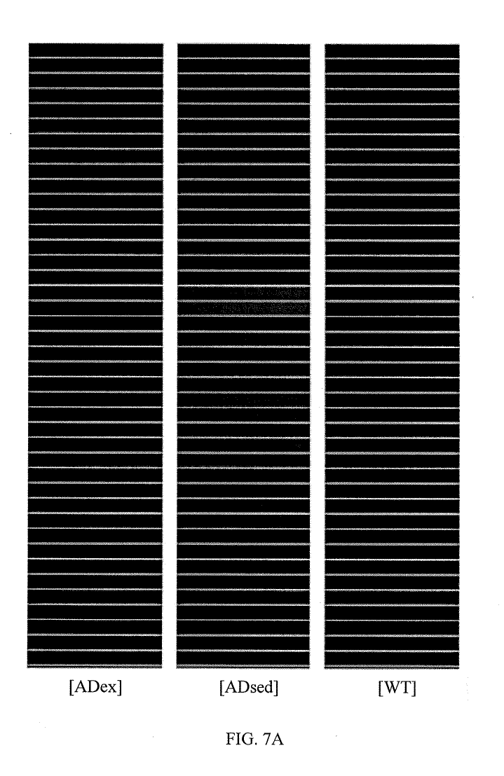

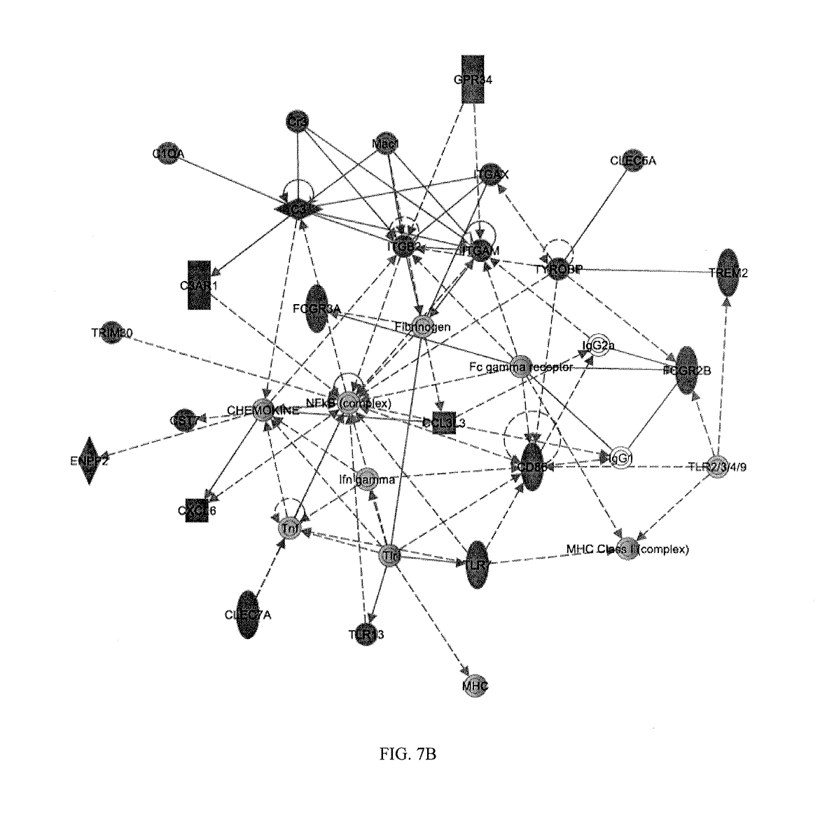

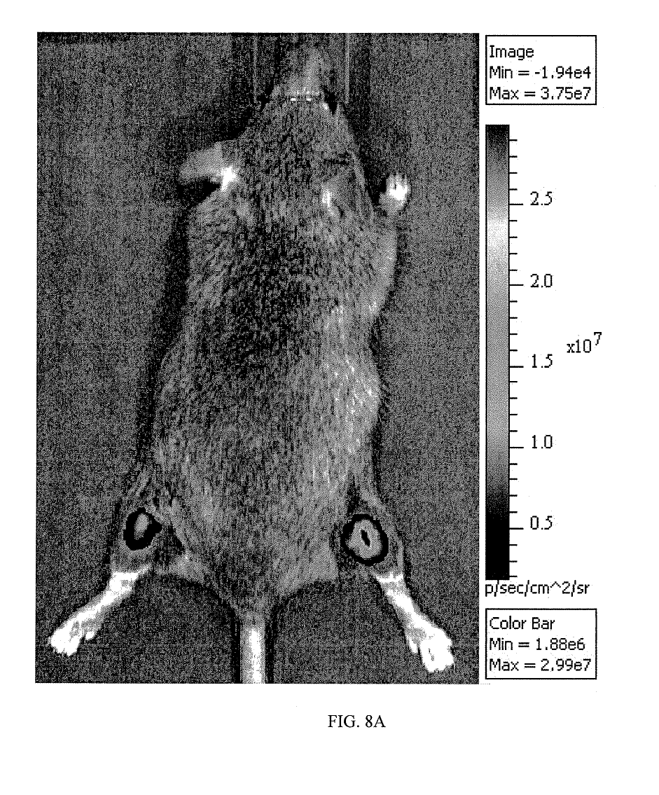

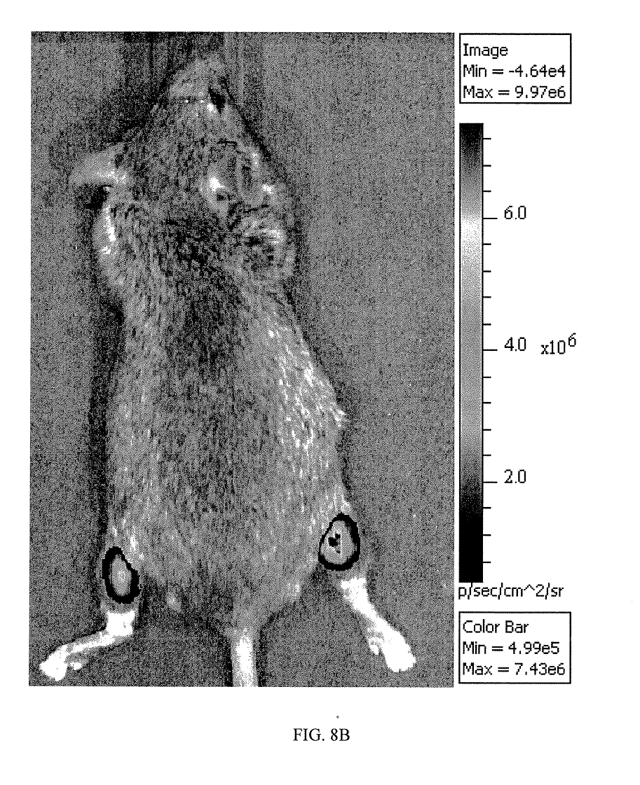

For these experiments, after sacrifice the brains were process for Aβ plaque immunostaining by thioflavine-S (as exemplified in FIG. 5). Around 100 five-μm slices were usually obtained from one hemisphere. Plaques were quantified for densitometry and count per mm² (Image-pro plus, Media Cybernetics). Further, soleus muscles were snap-frozen into liquid nitrogen and RNA was pooled for each animal and assayed by gene array (re: FIG. 7). Ideally, three gene chips were run using RNA pooled from three animals for each condition (total of 12), and qRT-PCR was performed to confirmed the change in expression for selected genes of interest.

Effect of Exercise on APP/PS1 Mice Behavior

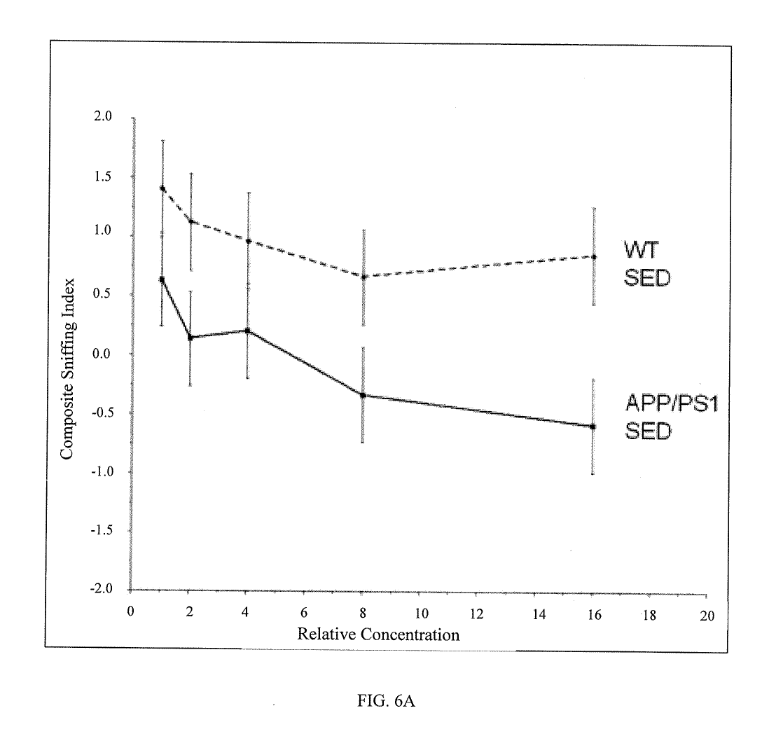

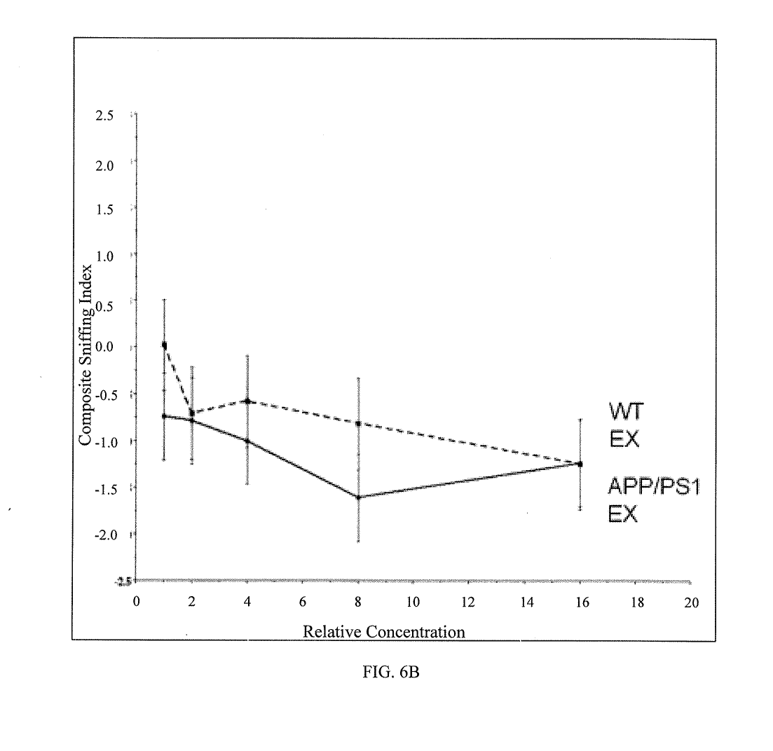

Olfactory dysfunction is a hallmark of AD and patients with early-stage exhibit deficits in the sense of smell, often before the appearance of overt memory loss. To examine the veracity of our model, it was examined whether, and to what degree APP/PS1 SED mice differed from WT SED controls on a test of olfactory function (n=4). Moreover, the lab examined the degree to which exercise ameliorated the effect (n=3). Briefly, an automated method for evaluating the responsivity to odorant stimuli was applied. Each animal had their stimulus-induced sniffing monitored in response to air and propanol odor, using whole-body plethysmography. For each animal a “Composite Sniffing Index” value was derived that incorporated 14 respiratory response measures in response to individual stimuli. These index values provided a univariate measure with which the response to propanol odor was evaluated as a function of genotype and exercise. There is a clear difference in the concentration response curves between APP/PS1 SED and their control counterpart (F=9.03; p<0.01). Importantly, exercise ameliorated this effect (FIG. 6) (F=0.4; p=0.52). The data also shows memory deficits consistent with AD. It was therefore demonstrated that APP/PS1 SED mice differ from WT SED on a novel object recognition task (n=8; t=0.18, p=0.86). Importantly, at 24 hours they differed in their retention of a conditioning object (n=8; t=−4.29, p=0.005). Thus, the experiments reveal an effective animal model to behaviorally evaluate the hypothesis.

Gene Profile in Skeletal Muscle of APP/PS1 Mice Following Exercise

To identify a gene candidate to treat the symptoms of the AD mouse model, gene profiling was performed (see, e.g., FIGS. 1 and 2). To date, no other study has assessed how exercise regulates gene expression in the muscle in the context of AD. The role of exercise will be reflected by the pattern of gene expression. A gene chip analysis was performed by pooling 3 soleus muscles (“SOL”) of different animals in each group and comparing APP/PS1 SED, EX, and WT. A mouse gene array (GeneChip 1.0 ST, Affymetrix) that will enable whole-genome assessment (28,853 genes) was used. RNA was isolated using RNeasy® Mini kit (QIAGEN) including the DNase treatment on the column, and gene arrays were performed by a microarray facility. Candidate genes revealed by GeneChip were confirmed using SYBR® GreenER™ Two-Step Universal Kit (Invitrogen), and qRT-PCR analysis was performed using a LightCycler® 480 Real-Time PCR System (Roche Applied Science).

The SOL was chosen because it is a postural muscle, among the first to be affected by exercise, and a mix of fibers that is a good representation of leg muscles. A total of 85 genes were upregulated and 306 were downregulated at least 1.5 fold by exercise in APP/PS1 mice. The results were analyzed using software that displays potential pathways (Ingenuity Pathway Analysis). Of the genes identified, 123 genes were involved in the inflammatory process and 28 were linked to the transcription factor NF-κB (FIG. 7). The data was confirmed using qRT-PCR in an array (SABiosciences) assessing genes dependent upon NF-κB pathway. The most upregulated gene was Interferon Releasing Factor-1 (“IRF-1”). Another gene identified was PPARγ coactivator 1-alpha (“PGC-1α”), a transcriptional coactivator which was of particular interest due to its role in biogenesis of mitochondria following exercise. PGC-1α was upregulated approximately 2.1 fold compared to sedentary animals. The data show that exercise induced gene regulation in muscle that is mostly involved in the inflammatory response in APP/PS mice.

Running Wheels for Exercise Protocol

The two common paradigms for a running wheel regimen are: i) regular sessions of forced running on a treadmill; and ii) free/voluntary access to a running wheel. Forced exercise has more variability in amelioration of cognition processes, whereas voluntary exercise produces more reliable neuroprotective effects. In addition, forced treadmill running can induce chronic stress, which could induce inflammation. Three weeks of running wheel exposure improves long/short-term memory in AD mice. APP/PS1 mice demonstrate a high level of exercise (Table 1).

TABLE 1
Physiological characteristics.

| | | Body weight | Soleus | | |
|---|---|---|---|---|---|
| | RPD | (g) | (mg) | EDL (mg) | GM (mg) |
| Exercise | 16234 ± | 39.4 ± 0.8 | 9.2 ± 0.3 | 10.6 ± 0.1 | 125.8 ± 10.2 |
| Sedentary | 4123 | 41.1 ± 0.5 | 8.1 ± 0.5 | 11.5 ± 0.2 | 120.2 ± 8.5 |

| Total Protein | Soleus (mg/mg) | EDL (mg/mg) | GM (mg/mg) |
|---|---|---|---|
| Exercise | 0.165 ± 0.006 | 0.155 ± 0.004 | 0.216 ± 0.010 |
| Sedentary | 0.170 ± 0.007 | 0.159 ± 0.004 | 0.221 ± 0.012 |

Rotation per day (RPD),
Extensor DigitorumLongus (EDL),
Gastrocnemius Mixed (GM)

Olfactory and Memory Tests

Each experimental session consists of monitoring the stimulus-induced sniffing response to propanol odor of age-matched APP/PS1 and WT male and female mice (SED and EX). The results, as exemplified in FIG. 6, are performed according to established methods. Following a habituation period, odors stimuli are randomly presented in 5 blocks of 20 trials, using an ascending series. Learning and memory deficits are tested using a novel object recognition task. Briefly, a mouse is placed into an open-air Rubbermaid container. During habituation, two of the three objects (marble pyramid, a Delrin disk and stainless-steel cube) are placed into the box equidistant from the center. The mouse is then placed in the box for 5 min. At 1 and 24 h following the habituation, two objects are placed into the box, one that was presented during habituation and one that is a “novel” object. Videos are then analyzed for recognition time.

### EXAMPLE 2

### Testing and Using Gene Candidates for Alzheimer's Disease

There is an abundant literature on gene delivery in the CNS. Using modified fibroblasts directly in the brain, gene encoding growth factors such as NGF and BDNF have been tested in rodent, primate models and Phase I clinical trials. Notwithstanding encouraging neuroprotective effects, the necessity of neuro-surgery involves obvious risks. Newly implanted cells could form tumors or migrate. Another approach is to use lentivirus to transfer genes to remotely access the brain. Studies by some have shown that expression of Neprilysin (a zinc metallopeptidase) specifically in leukocytes or muscle decreased brain Aβ peptide levels and deposits. Unlike antibodies, this method resulted in the catabolism of Aβ peptides, but long term adverse effects (i.e. immune reaction) associated with viruses are unknown and to date not practical for treating AD patients. The results described herein demonstrate that altering muscle gene expression could affect brain biochemistry and provide prophylaxis in the treatment of AD. This novel approach fundamentally broadens potential treatment in AD by placing emphasis on peripheral vis-à-vis the brain using muscle as a protein factory.

Skeletal Muscle Gene Delivery in vivo Monitoring

Typically, transgenic or KO animals are utilized to study gene function. However, AD is progressive and symptoms could occur 20 years after the disease onset. Thus, in vivo DNA transfer is therapeutically relevant because it is achievable at the time of a symptom's apparition. For example electroporation using the tweezer electrode method greatly increases gene transfer by briefly permeabilizing the cellular membrane and has been used efficiently in muscle.

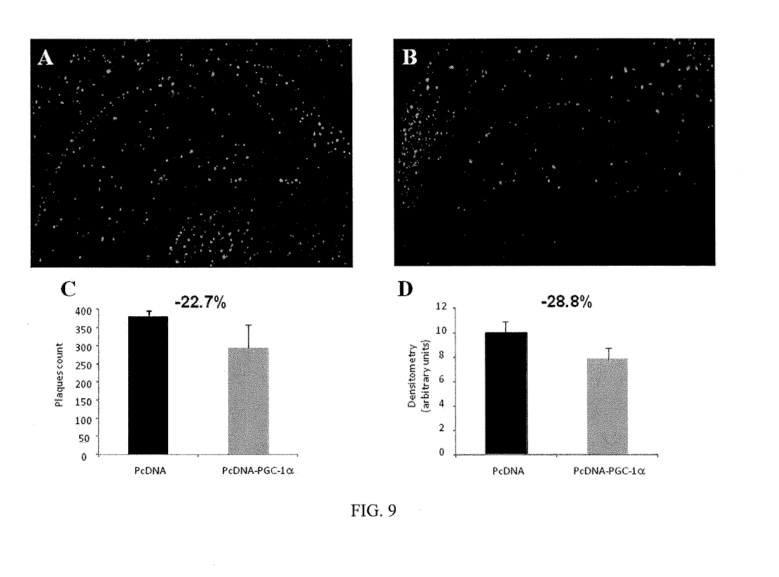

To evaluate the in vivo efficiency of the gene transfer, a mammalian expression plasmid (pCDNA) driven by a luciferase firefly construct was designed. Following injection of a substrate (D-Luciferin), the plasmid, if expressed, emits bioluminescence. The animals were then scanned with an imaging system (IVIS) and showed sustained activity for 30 days (FIG. 9), reproducing exercise timing on APP/PS1 mice where diminished plaque and behavioral improvement was previously demonstrated.

PGC-1α Overexpression in the Muscle Prevents AD-Associated Aβ Plaque

Using in vivo electroporation based on the results from the gene profile, IRF-1 and PGC-1α was overexpressed in the muscle of one year-old APP/PS1 mice. The plasmid was injected in the gastrocnemius (“GM”) and compared pCDNA (n=13) and pCDNA-PGC1α (n=13). Three animals were also injected with pCDNA-IRF-1, but only one survived after a month, perhaps due to the higher mortality of this mice model. The preliminary data show that overexpression of PGC-1α in the muscle reduced brain Aβ plaque number by 32.4% (P<0.005) and density by 25.3% (P<0.005) compared to APP/PS1 mice injected with pCDNA (FIG. 9). These results provide a strong rationale for the hypothesis, as they indicate that gene manipulation in muscle diminished Aβ plaque in the brain.

In Vivo Electroporation

For electroporation, 100 μg of expression grade DNA is injected into the GM (<100 -82 l). Electroporation is performed after injection (within 30 seconds). An electroporator (S48 Grass technologies, RI) is used in conjunction with “tweezertrodes” (Harvard Apparatus, MA) placed on each side of the muscle. The muscles receive 8 (20 ms) pulses of 200-V/cm at a frequency of 1 Hz. Based on the preliminary data and previous studies, it will not result in any trauma to either tissue or animal.

### EXAMPLE 3

### Treating Alzheimer's Disease in Humans According to One Embodiment

The methods described herein will be used to identify candidates for preventing and/or treating Alzheimer's disease (“AD”), especially since exercise is recognized as one of the most promising treatments for the disease, although an alternative to exercise is likely necessary as the majority of AD sufferers are age 65 and older, an age when other chronic diseases are likely to appear that limit exercise such as osteoarthritis and when instauration of a new lifestyle is more challenging. It is hypothesized that exercise induces changes in genes involved in the inflammatory process at the muscle level that protect the brain in AD, although the genes and pathways involved in this process are unknown. Further, it is hypothesized that gene delivery at the muscle level will partially reproduce the effect of exercise, including perhaps the anti-inflammatory effect of exercise, thereby reducing AD-associated biological and cognitive deficiencies.

Identifying Gene Candidates to Treat Cognitive Illness in Humans

To identify candidates for preventing and/or treating cognitive decline and/or the symptoms of cognitive decline (including, for example, Alzheimer's disease) in humans, a method as described herein will be used. For example, the method described in FIGS. 1 and 2 can be used. At step 10, AD-sufferers (and/or non-AD sufferers) are exposed to either a “normal” regimen, a sedentary regimen, or an exercise regimen (increased physical activity compared to normal). The exercise regimen can be, for example, anywhere along a scale from extremely intense to extremely moderate or weak, depending upon the requirements of the experiments and the abilities of the subject. The regimens can be implemented for a variable amount of time ranging from minutes to months or years.

At step 12, RNA is obtained from muscle cells in each regimen group. For example, RNA can be isolated from cells obtained from an individual in the exercise regimen group and an individual in the non-exercise regimen group. At step 14, gene profiling is performed using the obtained RNA according to standard protocols known in the art. At step 16, the genes whose expression is either upregulated or downregulated in the exercise regimen group compared to the non-exercise regimen group are identified. One or more of these genes are then examined according to other methods described herein to determine the viability of that gene to treat the condition or disease of interest.

As above, at optional step 28, the desired phenotype is confirmed by comparing the phenotypes of mice in the different groups. For example, the people in the exercise group are compared to people in a non-exercise group to determine whether there is an improvement in one or more hallmarks of cognition or other behavior following exercise.

Using Identified Gene Candidates in Humans to Treat Cognitive Problems

To use identified candidates to prevent and/or treat cognitive decline and/or the symptoms of cognitive decline (including, for example, Alzheimer's disease) in humans, a method as described herein will be used. For example, the method described in FIGS. 3 and 4 can be used. At step 30, the candidate gene is identified using any of the methods identified herein, including those depicted in FIGS. 1 and 2. In one embodiment, the gene is identified as one that is significantly up-regulated or down-regulated during conditions that reduce the effect of the disease (such as exercise, etc.).

Alternatively, the candidate can be identified using a variety of other means, including but not limited to the following: (i) gene profiling of obese versus non-obese people to identify candidates for obesity treatments; (ii) gene profiling of arthritic versus non-arthritic people to identify candidates for arthritis treatments; (iii) gene profiling of plaque-covered versus non-plaque-covered arterial cells for hypertension and/or artherosclerosis treatments; (iv) gene profiling of cancer versus non-cancer cells; and (v) gene profiling of insulin-producing cells in diabetic versus non-diabetic or pre-diabetic patients. Many other methods of identifying a treatment candidate are possible.

At step 32 the gene (or, perhaps, gene repressor) is transfected into the human, where transfection is the introduction of a nucleic acid or other agent into one or more cells. This may be transfection of the candidate gene, or may be transfection of an agent that affects the candidate (such as, for example, an activator or inhibitor of either expression or translation). In a preferred embodiment, the nucleic acid or other agent is transfected into a peripheral cell. In one embodiment, described in detail below, the identified gene candidate is introduced into peripheral cells, such as muscle tissue, by electroporation, although other methods of transfection are known.

At optional step 36, the effect of the activated element on the disease is analyzed. This step confirms that the candidate was properly identified, and that inducing or inhibiting the expression of the candidate is able to effectuate a disease treatment. In one example, studies are conducted to determine whether expression of the gene candidate mimics the effect of intense exercise (including Aβ plaque reduction and the amelioration of memory and olfactory deficits). If expression of the candidate does mimic the effects of intense exercise, then the candidate was properly identified and is thus a viable candidate for treatment.

Studies will likely determine that PGC-1α is a viable candidate in humans for treatment of one or more conditions such as cognitive decline (including Alzheimer's disease), just as it is in mice. Similar to FIG. 4, the PGC-1α gene will be inserted into a suitable vector for human transfection (a method similar to the electroporation in the mouse model can be used to transfect the gene and/or vector). Notably, the PGC-1α gene may have to be adapted, for example, to improve transcription and/or function of the transcribed protein, among other adaptations. Once transfected into human muscle cells, for example, the overexpression of transcription factor PGC-1α will affect a variety of other proteins (potentially by affecting—either by increasing or decreasing—transcription of one or more genes in the genome, or by interacting with other proteins in the cell to affect one or more cellular pathways). This cascade will ultimately result in increased mitochondria biogenesis and improvement in one or more hallmarks of cognitive decline/AD. This represents one of the first methods or mechanisms for treating AD or cognitive decline using a peripheral system; that is, treating AD without specifically targeting brain tissue directly.

### EXAMPLE 4

### Treating Other Conditions/Diseases in Humans According to One Embodiment

To identify candidates for preventing and/or treating other illnesses in humans, one or more methods as described herein will be used. For example, the method described in FIGS. 1 and 2 can be used. At step 10, sufferers are exposed to either a “normal” regimen (sedentary or normal physical activity) or an exercise regimen (increased physical activity compared to normal). The exercise regimen can be, for example, anywhere along a scale from extremely intense to extremely moderate or weak, depending upon the requirements of the experiments and the abilities of the subject. The non-exercise regimen can be anywhere along a scale of bed-ridden to moderate exercise regimens. The regimens can be implemented for a variable amount of time ranging from minutes to months or years.

At step 12, RNA is obtained from muscle cells from one or more individuals in each regimen group. For example, RNA can be isolated from cells obtained from an individual in the exercise regimen group and an individual in the non-exercise regimen group. At step 14, gene profiling is performed using the obtained RNA according to standard protocols known in the art. At step 16, the genes whose expression is either upregulated or downregulated in the exercise regimen group compared to the non-exercise regimen group are identified. One or more of these genes are then examined according to other methods described herein to determine the viability of that gene to treat the condition or disease of interest.

As above, at optional step 28, the desired phenotype is confirmed by comparing the phenotypes of mice in the different groups. For example, the people in the exercise group are compared to people in a non-exercise group to determine whether there is an improvement in one or more hallmarks of the disease or condition of interest following exercise.

Using Identified Gene Candidates in Humans to Treat the Condition/Disease

To use identified candidates to prevent and/or treat the condition or disease of interest, one or more of the methods as described herein will be used. For example, the methods described in FIGS. 3 and 4 can be used. At step 30, the candidate gene is identified using any of the methods identified herein, including those depicted in FIGS. 1 and 2. In one embodiment, the gene is identified as one that is significantly up-regulated or down-regulated during conditions that reduce the effect of the disease (such as exercise, etc.).

Alternatively, the candidate can be identified using a variety of other means, including but not limited to the following: (i) gene profiling of obese versus non-obese people to identify candidates for obesity treatments; (ii) gene profiling of arthritic versus non-arthritic people to identify candidates for arthritis treatments; (iii) gene profiling of plaque-covered versus non-plaque-covered arterial cells for hypertension and/or artherosclerosis treatments; (iv) gene profiling of cancer versus non-cancer cells; and (v) gene profiling of insulin-producing cells in diabetic versus non-diabetic or pre-diabetic patients. Many other methods of identifying a treatment candidate are possible.

At step 32 the gene (or, perhaps, gene repressor) is transfected into the human, where transfection is the introduction of a nucleic acid or other agent into one or more cells. This may be transfection of the candidate gene, or may be transfection of an agent that affects the candidate (such as, for example, an activator or inhibitor of either expression or translation). In a preferred embodiment, the nucleic acid or other agent is transfected into a peripheral cell. In one embodiment, described in detail below, the identified gene candidate is introduced into peripheral cells, such as muscle tissue, by electroporation, although other methods of transfection are known. At optional step 36, the effect of the activated element on the disease is analyzed. This step confirms that the candidate was properly identified, and that inducing or inhibiting the expression of the candidate is able to effectuate a disease treatment. If expression of the candidate does mimic the effects of intense exercise, then the candidate was properly identified and is thus a viable candidate for treatment.

Although the present invention has been described in connection with a preferred embodiment, it should be understood that modifications, alterations, and additions can be made to the invention without departing from the scope of the invention as defined by the claims.

What is claimed is:

1. A method of identifying an agent for the treatment of a disease in a subject, comprising the steps of:
exposing the subject to an exercise regimen, wherein said exercise regimen results in an improvement of a hallmark of said disease;
profiling the expression of a gene in said subject; and
determining whether the expression of said gene is altered after exposure to said exercise regimen.

2. The method of claim 1, wherein the expression of said gene is up-regulated after exposure to said exercise regimen.

3. The method of claim 1, wherein the expression of said gene is down-regulated after exposure to said exercise regimen.

4. The method of claim 1, further comprising the steps of:
generating a vector comprising at least a portion of said identified gene; and
transfecting said vector into a cell of a second subject.

5. The method of claim 4, further comprising the step of:
determining whether transfection of said vector into a cell of a second subject results in an improvement of a hallmark of said disease.

6. The method of claim 4, wherein said cell is remote from the brain of said subject.

7. The method of claim 6, wherein said cell is a muscle cell.

8. The method of claim 1, wherein said subject is a mammal.

9. The method of claim 8, wherein said mammal is a human.

10. A method for the treatment of a condition, said method comprising the steps of:
identifying a target for the treatment of said condition, wherein said target is identified by a method comprising the steps of: (i) exposing a first subject to an exercise regimen, wherein said exercise regimen results in an improvement of a hallmark of said condition; (ii) profiling the expression of a gene in said first subject; and (iii) identifying said gene as said target if said expression is altered after exposure to said exercise regimen;
generating a vector comprising said identified gene; and
transfecting said vector into a cell of a subject suffering from said condition.

11. The method of claim 10, wherein the expression of said gene is up-regulated after exposure to said exercise regimen.

12. The method of claim 10, wherein the expression of said gene is down-regulated after exposure to said exercise regimen.

13. The method of claim 10, wherein said cell is remote from the brain.

14. The method of claim 13, wherein said cell is a muscle cell.

15. The method of claim 10, wherein said subject is a mammal.

16. The method of claim 15, wherein said mammal is a human.

17. The method of claim 10, wherein said condition is selected from the group consisting of: dementia, obesity, and diabetes.

18. The method of claim 10, wherein said condition is Alzheimer's disease.

19. A method of treating cognitive decline in a subject using PPARγ coactivator 1-alpha (“PGC-1α”) protein, said method comprising the step of expressing a gene sequence in a cell of said subject whereby said PGC-1α protein is produced.

20. The method of claim 19, wherein said cell is remote from the brain of said subject.

21. The method of claim 20, wherein said cell is a muscle cell.

22. A method of treating cognitive decline in a subject using PPARγ coactivator 1-alpha (“PGC-1α”) protein, said method comprising the step of increasing an activity of PGC-1α in a cell of said subject.

23. The method of claim 22, wherein said cell is remote from the brain of said subject.

24. The method of claim 23, wherein said cell is a muscle cell.

25. The method of claim 22, wherein increasing an activity of PGC-1α comprises increasing the expression of PGC-1α in a cell of said subject.
